(19) United States
(12) Patent Application Publication
(10) Pub. No.: US 2010/0197956 A1
(43) Pub. Date: Aug. 5, 2010

(54) Vam Shell Catalyst, Method For Its Production And Use Thereof

(75) Inventors: Alfred Hagemeyer, Bad Aibling (DE); Gerhard Mestl, Munich (DE); Peter Scheck, Gilching (DE); Alice Kyriopoulos, Holzkirchen (DE)

Correspondence Address: RATNERPRESTIA, P.O. BOX 980, VALLEY FORGE, PA, 19482, US

(73) Assignee: SUD-CHEMIE AG, Munich (DE)

(21) Appl. No.: 12/602,315

(22) PCT Filed: May 30, 2008
(86) PCT No.: PCT/EP2008/004329

§ 371 Date: Apr. 21, 2010

(30) Foreign Application Priority Data
May 31, 2007 (DE) 10 2007 025 444.1

(51) Int. Cl. B01J 21/16; B01J 31/02; C07C 67/00; C07C 67/39

(52) U.S. Cl. 560/208; 502/84; 502/74; 502/62; 560/205

(57) ABSTRACT

A shell catalyst for the production of vinyl acetate monomer (VAM), comprising a porous catalyst support based on a natural sheet silicate, in particular based on an acid-treated calcined bentonite, said catalyst support being loaded with Pd and Au and being designed as a shaped body. In order to provide a shell catalyst for the production of VAM, which shell catalyst is characterized by a relatively high VAM selectivity and also a high activity, it is proposed that the catalyst support has a surface area of less than 130 m²/g.

### CROSS REFERENCE TO RELATED APPLICATIONS

This is a National Phase application of PCT application number PCT/EP2008/004329, filed May 30, 2009, which claims priority benefit of German application number DE 10 2007 025 444.1, filed May 31, 2007, the content of such applications being incorporated herein by reference.

### FIELD OF THE INVENTION

The present invention relates to a shell catalyst for the production of vinyl acetate monomer (VAM), comprising a porous catalyst support based on a natural sheet-sheet-silicate, in particular based on an acid-treated calcined bentonite, said catalyst support being loaded with Pd and Au and being designed as a shaped body.

### BACKGROUND OF THE INVENTION

VAM is an important monomer building block in the synthesis of plastic polymers. The main fields of use of VAM are, inter alia, the production of polyvinyl acetate, polyvinyl alcohol and polyvinyl acetal and also copolymerisation and terpolymerisation with other monomers such as, for example, ethylene, vinyl chloride, acrylate, maleinate, fumarate and vinyl laurate.

VAM is produced primarily in the gas phase from acetic acid and ethylene by reaction with oxygen, wherein the catalysts used for this synthesis preferably contain Pd and Au as active metals and also an alkali metal component as promoter, preferably potassium in the form of the acetate. In the Pd/Au system of these catalysts, the active metals Pd and Au are assumed to exist not in the form of metal particles of the respective pure metal but rather in the form of Pd/Au alloy particles of possibly varying composition, although the presence of unalloyed particles cannot be ruled out. As an alternative to Au, use may also be made for example of Cd or Ba as the second active metal component.

At present, VAM is produced mainly by means of so-called shell catalysts, in which the catalytically active metals of the catalyst do not completely penetrate the catalyst support designed as a shaped body but rather are contained in a more or less broad outer region (shell) of the catalyst support shaped body (cf. in this regard EP 565 952 A1, EP 634 214 A1, EP 634 209 A1 and EP 634 208 A1), whereas the more inward-lying regions of the support are almost free of noble metal. Using shell catalysts, it is possible in many cases for the reaction to proceed more selectively than with catalysts in which the supports are impregnated with the active components right into the support core (“thoroughly impregnated”).

The shell catalysts known in the prior art for the production of VAM may be for example catalyst supports based on silicon oxide, aluminium oxide, aluminosilicate, titanium oxide or zirconium oxide (cf. in this regard EP 839 793 A1, WO 1998/018553 A1, WO 2000/058008 A1 and WO 2005/061107 A1). However, catalyst supports based on titanium oxide or zirconium oxide are hardly used at present since these catalyst supports are not durably stable with respect to acetic acid and are relatively expensive.

Most of the catalysts used at present for the production of VAM are shell catalysts comprising a Pd/Au shell on a porous, amorphous, spherical aluminosilicate support based on natural sheet-sheet-silicates, in particular based on natural acid-treated calcined bentonites which are thoroughly impregnated with potassium acetate as promoter.

Such VAM shell catalysts are usually produced via the so-called chemical route, in which the catalyst support is impregnated with solutions of suitable metal precursor compounds, for example by dipping the support into the solutions or by means of the incipient wetness method (pore filling method), in which the support is loaded with a volume of solution corresponding to its pore volume. The Pd/Au shell of the catalyst is produced for example by firstly impregnating the catalyst support shaped body in a first step with an Na₂PdCl₄ solution and then, in a second step, using NaOH solution to fix the Pd component on the catalyst support in the form of a Pd hydroxide compound. In a subsequent, separate third step, the catalyst support is then impregnated with an NaAuCl₄ solution and thereafter the Au component is likewise fixed by means of NaOH. After fixing the noble metal components in an outer shell of the catalyst support, the loaded catalyst support is then washed until it is largely free of chloride and Na ions, then dried and finally reduced with ethylene at 150° C. The Pd/Au shell produced usually has a thickness of approximately 100 to 500 μm.

After the fixing or reduction step, the catalyst support loaded with the noble metals is usually loaded with potassium acetate, wherein the loading with potassium acetate does not take place only in the outer shell loaded with noble metals but rather the catalyst support is completely thoroughly impregnated with the promoter. As the catalyst support, use is predominantly made of a spherical support bearing the reference “KA-160” from SÜD-Chemie AG based on natural acid-treated bentonites as the natural sheet-sheet-silicate, which has a BET surface area of approximately 160 m²/g.

The VAM selectivities achieved by the shell catalysts known from the prior art based on Pd and Au as active metals and KA-160 supports as catalyst supports are approximately 90 mol % relative to the amount of ethylene supplied, with the remaining 10 mol % of the reaction products being substantially CO₂, which is formed by total oxidation of the organic reagents/products.

An increase in the VAM selectivity is desirable in order to reduce the costs for raw material losses and to make it easier and therefore less expensive to prepare the reaction product VAM.

### SUMMARY OF THE INVENTION

An object of the present invention is therefore to provide a shell catalyst for the production of VAM, which is characterised by a relatively high VAM selectivity and also a high activity.

Starting from a shell catalyst of the generic type, this object is achieved in that the catalyst support has a surface area of less than 130 m²/g.

The invention therefore relates to a shell catalyst comprising a natural sheet-sheet-silicate, in particular to a catalyst support shaped body comprising an acid-treated calcined bentonite, having an outer shell which contains Pd and Au in metallic form, the catalyst support shaped body having a BET surface area of less than 130 m²/g.

Shell catalysts comprising a support with an outer shell into which the active species has penetrated are also referred to in the prior art as “egg shell” shell catalysts.

It has surprisingly been found that the shell catalyst according to aspects of the invention is characterised by a VAM selectivity which is at least 1 mol % higher that that of the corresponding catalysts known in the prior art for the production of VAM. The increase in selectivity can essentially be attributed to a reduction in the undesired total oxidation of acetic acid, ethylene and VAM to form CO₂.

The catalyst according to aspects of the invention has an activity that is at least as high as that of the corresponding catalysts known in the prior art for the production of VAM. Furthermore, it has been found that the activity of the catalyst according to aspects of the invention can be considerably increased by increasing the thickness of the Pd/Au shell, without having to accept appreciable losses in VAM selectivity. In the corresponding catalysts known in the prior art, an increase in shell thickness is associated with a considerably reduced VAM selectivity.

Furthermore, despite its relatively small surface area, i.e. despite its relatively large pore volume, the catalyst according to aspects of the invention has excellent mechanical stability and exhibits high chemical resistance against the reagents and products to be used and also high thermal resistance against the temperatures used in VAM synthesis.

If the reaction conditions for industrial use of the catalyst according to the aspects of the invention are left unchanged compared to a corresponding shell catalyst of the prior art, it is thus possible to produce more VAM per reactor volume and per unit time, which equates to an increase in capacity and also additional expenditure. Furthermore, it is easier to work up the resulting crude vinyl acetate since the VAM content in the product gas is higher, which leads to an energy saving during VAM work-up. Suitable work-up methods are disclosed for example in U.S. Pat. No. 5,066,365 A and DE 29 45 913 A1.

If, on the other hand, the VAM production capacity of an installation loaded with the catalyst according to the aspects of the invention is kept constant at the level of a corresponding known shell catalyst, then the reaction temperature can be lowered when using the catalyst according to the aspects of the invention, as a result of which a further increase in VAM selectivity can be obtained along with the advantageous effects mentioned above. In this case, the amount of CO₂ which results as a by-product and which therefore has to be removed is lower, as is the loss of entrained ethylene associated with this removal. Furthermore, such a way of carrying out the method on a corresponding installation leads to an extended life of the catalyst due to lower temperatures.

The expression “based on a natural sheet-sheet-silicate” is understood here to mean that the catalyst support shaped body comprises a natural sheet-sheet-silicate, wherein the natural sheet-sheet-silicate may be contained in the catalyst support either in an untreated or in a treated form. Typical treatments to which a natural sheet-sheet-silicate may be subjected prior to being used as a support material include for example treatment with acids and/or calcination. In the context of the present invention, the term “natural sheet-sheet-silicate” is understood to mean silicate material originating from natural sources and in which SiO₄ tetrahedra, which form the structural basic unit of all silicates, are crosslinked to one another in layers of general formula [Si₂O₅]²⁻. These layers of tetrahedra alternate with layers of so-called octahedra, in which a cation, especially Al and Mg, is surrounded by OH and/or O in the shape of an octahedron. A distinction is made for example between two-layer sheet-sheet-silicates and three-layer sheet-sheet-silicates.

Sheet-Sheet-silicates which are preferred in the context of the present invention are clay minerals, in particular kaolinite, beidellite, hectorite, saponite, nontronite, mica, vermiculite and smectites, with particular preference being given to smectites and in particular to montmorillonite. Definitions in German of the term “sheet-silicates” can be found for example in “Lehrbuch der anorganischen Chemie”, Hollemann Wiberg, de Gruyter, 102nd edition, 2007 (ISBN 978-3-11-017770-1) or in “Römpp Lexikon Chemie”, 10th edition, Georg Thieme Verlag under the term “Sheet-silikat”. One natural sheet-silicate which is particularly preferred in the context of the present invention is a bentonite. Bentonites are not natural sheet-silicates in the actual sense of the word but are instead a mixture of mainly clay minerals containing sheet-silicates. Therefore, in the case where the natural sheet-silicate here is a bentonite, it is to be understood that the natural sheet-silicate in the catalyst support is present in the form of or as a constituent of a bentonite.

It has been found that the smaller the surface area of the catalyst support, the higher the VAM selectivity of the catalyst according to the aspects of the invention. Furthermore, the smaller the surface area of the catalyst support, the more the thickness of the Pd/Au shell can be selected to be greater, without any appreciable losses of VAM selectivity ensuing. According to one preferred embodiment of the catalyst according to the aspects of the invention, the surface area of the catalyst support has a size of less than 125 m²/g, preferably less than 120 m²/g, more preferably less than 100 m²/g, even more preferably less than 80 m²/g and particularly preferably less than 65 m²/g. In the context of the present invention, the term “surface area” of the catalyst support is understood to mean the BET surface area of the support, which is determined by nitrogen adsorption according to DIN 66132.

According to another preferred embodiment of the catalyst according to the aspects of the invention, it may be provided that the catalyst support has a surface area of between 130 and 40 m²/g, preferably between 128 and 50 m²/g, more preferably between 126 and 50 m²/g, even more preferably between 125 and 50 m²/g, still more preferably between 120 and 50 m²/g and most preferably between 100 and 60 m²/g.

A catalyst support designed as a shaped body and based on natural sheet-silicates, in particular based on an acid-treated calcined bentonite, wherein the catalyst support has a surface area of less than 130 m²/g, preferably a surface area of between 130 and 40 m²/g, may be produced for example by moulding a moulding mixture containing an acid-treated (uncalcined) bentonite as sheet-silicate and water, by compression, to form a shaped body using devices familiar to the person skilled in the art, such as extruders or tablet presses for example, and then the uncured shaped body is calcined to form a stable shaped body. Here, the size of the specific surface area of the catalyst support depends in particular on the quality of the (raw) bentonite used, the acid treatment method for the bentonite used, i.e. for example the type and quantity (relative to the bentonite) and concentration of the inorganic acid used, the acid treatment time and temperature, the compression force and also the calcination time and temperature and the calcination atmosphere. A suitable catalyst support having a surface area of approximately 100 m²/g is sold by SÜD-Chemie AG under the name “KA-0”.

Acid-treated bentonites can be obtained by treating bentonites with strong acids, such as sulphuric acid, phosphoric acid or hydrochloric acid for example. A German definition of the term “bentonite” which also applies in the context of the present invention is given in Römpp, Lexikon Chemie, 10th edition, Georg Thieme Verlag. Bentonites which are particularly preferred in the context of the present invention are natural aluminium-containing sheet-silicates which contain montmorillonite (as smectite) as the main mineral. After the acid treatment, the bentonite is usually washed with water, dried and ground to a powder.

The acidity of the catalyst support may advantageously influence the activity of the catalyst according to the aspects of the invention in the gas-phase synthesis of VAM from acetic acid and ethylene. According to another preferred embodiment of the catalyst according to the aspects of the invention, the catalyst support has an acidity of between 1 and 150 μeq/g, preferably between 5 and 130 μeq/g, more preferably between 10 and 100 μeq/g and particularly preferably between 10 and 60 μeq/g. The acidity of the catalyst support is determined here as follows: 1 g of the finely ground catalyst support is mixed with 100 ml of water (with a pH blank value) and extracted for 15 minutes with stirring. Titration is then carried out using 0.01 n NaOH solution at least until pH 7.0 is obtained, with the titration taking place in stages; specifically, firstly 1 ml of the NaOH solution is added dropwise to the extract (1 drop/second), then there is a wait of 2 minutes, the pH is read, then another 1 ml of NaOH is added dropwise, and so on. The blank value of the water used is determined and the acidity calculation is corrected accordingly.

The titration curve (ml 0.01 NaOH against pH) is then plotted and the point of intersection of the titration curve at pH 7 is determined. The molar equivalents are calculated in 10⁻⁶ eq/g of support, resulting from the NaOH consumption for the point of intersection at pH 7.

Total acid: (10* ml 0.01 n NaOH)/1 support=μeq/g

With regard to a low pore diffusion limitation, it may be provided according to another preferred embodiment of the catalyst according to the aspects of the invention that the catalyst support has an average pore diameter of 8 to 50 nm, preferably 10 to 35 nm and more preferably 11 to 30 nm.

The catalyst according to the aspects of the invention is usually produced by subjecting a large number of catalyst support shaped bodies to a “batch” method, during the individual method steps of which the shaped bodies are subjected to relatively high mechanical loads applied for example by means of stirring and mixing tools. Furthermore, the catalyst according to the aspects of the invention may be subjected to considerable mechanical stress during the filling of a reactor, which may lead to the undesirable creation of dust and to damage to the catalyst support, in particular to its catalytically active shell located in an outer region. Particularly with the aim of keeping the abrasion of the catalyst according to the aspects of the invention within reasonable limits, the catalyst has a hardness of greater than/equal to 20 N, preferably greater than/equal to 25 N, more preferably greater than/equal to 35 N and most preferably greater than/equal to 40 N. The hardness is determined using a tablet hardness tester 8M from the company Dr. Schleuniger Pharmatron AG as an average over 99 shell catalysts after drying the catalyst at 130° C. for 2 h, with the device settings being as follows:

| Hardness: | N | |
|---|---|---|
| Distance from the shaped body: | 5.00 | mm |
| Time delay: | 0.80 | s |
| Type of advance: | 6 | D |
| Speed: | 0.60 | mm/s |

The hardness of the catalyst or catalyst support can be influenced for example by varying certain parameters of the method for its production, for example through the choice of sheet-silicate, the calcination time and/or the calcination temperature for an uncured shaped body formed from a suitable support mixture, or by means of certain additives such as methylcellulose or magnesium stearate for example.

The catalyst according to the aspects of the invention comprises a catalyst body designed as a shaped body and based on a natural sheet-silicate, in particular based on an acid-treated calcined bentonite. In the context of the present invention, the expression “based on” means that the catalyst comprises a natural sheet-silicate. It may be preferred if the content of natural sheet-silicate, in particular acid-treated calcined bentonite, in the catalyst support is greater than/equal to 50% by weight, preferably greater than/equal to 60% by weight, more preferably greater than/equal to 70% by weight, even more preferably greater than/equal to 80% by weight, still more preferably greater than/equal to 90% by weight and most preferably greater than/equal to 95% by weight, relative to the weight of the catalyst support.

It has been found that the VAM selectivity of the catalyst according to the aspects of the invention depends on the integral pore volume of the catalyst support. It is preferred if the catalyst support has an integral pore volume according to BJH of between 0.25 and 0.7 ml/g, preferably between 0.3 and 0.6 ml/g and more preferably from 0.35 to 0.5 ml/g. Here, the integral pore volume of the catalyst support is determined by means of nitrogen adsorption in accordance with the BJH method. The surface area of the catalyst support and its integral pore volume are determined in accordance with the BET and BJH method respectively. The BET surface area is determined in accordance with the BET method according to DIN 66131; the BET method is also published in J. Am. Chem. Soc. 60, 309 (1938). In order to determine the surface area and integral pore volume of the catalyst support or catalyst, the sample can be measured for example using a fully automatic nitrogen porosimeter from the company Micromeritics, type ASAP 2010, by means of which an adsorption and desorption isotherm is recorded.

In order to determine the surface area and porosity of the catalyst support or catalyst according to the BET theory, the data are evaluated according to DIN 66131. The pore volume is determined from the measurement data using the BJH method (E. P. Barret, L. G. Joiner, P. P. Haienda, J. Am. Chem. Soc. 73 (1951, 373)). This method also takes account of the effects of capillary condensation. Pore volumes of certain pore size ranges are determined by summing incremental pore volumes obtained from the evaluation of the adsorption isotherm according to BJH. The integral pore volume according to the BJH method relates to pores having a diameter of 1.7 to 300 nm.

According to another preferred embodiment of the catalyst according to the aspects of the invention, it may be provided that the water absorbency of the catalyst support is 40 to 75%, preferably 50 to 70%, calculated as the increase in weight due to water absorption. The absorbency is determined by impregnating 10 g of the support sample with deionised water for 30 min, until no more gas bubbles leave the support sample. The excess water is then decanted and the impregnated sample is dabbed with a cotton cloth to remove any adhering moisture from the sample. The support loaded with water is then weighed and the absorbency is calculated as follows:

(end weight (g)−initial weight (g))×10=water absorbency (%)

According to another preferred embodiment of the catalyst according to the aspects of the invention, it may be preferred if at least 80% of the integral pore volume of the catalyst support according to BJH is formed of mesopores and macropores, preferably at least 85% and more preferably at least 90%. This counteracts any reduced activity of the catalyst according to the aspects of the invention brought about through diffusion limitation, particularly in the case of Pd/Au shells with relatively large thicknesses. Here, the terms micropores, mesopores and macropores should be understood to mean pores which have a diameter of less than 2 nm, a diameter of 2 to 50 nm and a diameter of more than 50 nm, respectively.

The catalyst support of the catalyst according to the aspects of the invention may have a bulk density of more than 0.3 g/ml, preferably more than 0.35 g/ml and particularly preferably a bulk density of between 0.35 and 0.6 g/ml.

In order to ensure sufficient chemical resistance of the catalyst according to the aspects of the invention, the natural sheet-silicate contained in the support has an SiO₂ content of at least 65% by weight, preferably at least 80% by weight and more preferably from 95 to 99.5% by weight, relative to the weight of the sheet-silicate.

In the gas-phase synthesis of VAM from acetic acid and ethylene, a relatively low Al₂O₃ content in the sheet-silicate rarely has a disadvantageous effect, whereas a marked reduction in hardness has to be taken into account at high Al₂O₃ contents. Therefore, according to a preferred embodiment of the catalyst according to the aspects of the invention, the sheet-silicate contains less than 10% by weight of Al₂O₃, preferably 0.1 to 3% by weight and more preferably 0.3 to 1.0% by weight, relative to the weight of the sheet-silicate.

The catalyst support of the catalyst according to the aspects of the invention is designed as a shaped body. The catalyst support may in principle take the shape of any geometric body to which a suitable noble metal shell can be applied. However, it is preferred if the catalyst support is shaped as a sphere, cylinder (including with rounded end faces), perforated cylinder (including with rounded end faces), triple lobe, “capped tablet”, quadruple lobe, ring, doughnut, star, cartwheel, “inverse” cartwheel, or as a strand, preferably as a ribbed strand or star-shaped strand, preferably as a sphere.

The diameter and/or length and thickness of the catalyst support of the catalyst according to the aspects of the invention is preferably 2 to 9 mm, depending on the geometry of the reactor tube in which the catalyst is to be used. If the catalyst support is shaped as a sphere, the catalyst support preferably has a diameter of more than 2 mm, preferably a diameter of more than 3 mm and more preferably a diameter of more than 4 mm to 9 mm.

In order to increase the activity of the catalyst according to the aspects of the invention, it may be provided that the catalyst support is doped with at least one oxide of a metal selected from the group consisting of Zr, Hf, Ti, Nb, Ta, W, Mg, Re, Y and Fe, preferably with ZrO₂, HfO₂ or Fe₂O₃. It may be preferred if the content of dopant oxide in the catalyst support is between 0.01 and 20% by weight, preferably 1.0 to 10% by weight and more preferably 3 to 8% by weight, relative to the weight of the catalyst support. The quantity of dopant oxide depends primarily on the type of dopant oxide being used.

In general, the smaller the thickness of the Pd/Au shell, the higher the VAM selectivity of the catalyst according to the aspects of the invention. According to another preferred embodiment of the catalyst according to the aspects of the invention, therefore, the shell of the catalyst has a thickness of less than 300 μm, preferably less than 200 μm, more preferably less than 150 μm, even more preferably less than 100 μm and still more preferably less than 80 μm. The thickness of the shell can be measured optically using a microscope. Specifically, the region in which the noble metals are deposited appears black, whereas the regions which are free of noble metals appear white. The boundary line between the regions containing noble metals and the regions which are free of noble metals is usually very sharp and clearly visible. If the aforementioned boundary line should not be sharp and accordingly clearly visible, then the thickness of the shell corresponds to the thickness of a shell, measured from the outer surface of the catalyst support, which contains 95% of the noble metal deposited on the support.

However, it has also been found that, in the catalyst according to the aspects of the invention, the Pd/Au shell (as a function of the BET surface area of the support) can be formed with a relatively large thickness giving rise to a high activity of the catalyst, without causing any appreciable reduction in the VAM selectivity of the catalyst according to the aspects of the invention. In this case, the thickness of the noble metal shell can increase in an approximately inversely proportional manner to the BET surface area of the catalyst support. According to another preferred embodiment of the catalyst according to the aspects of the invention, the shell of the catalyst therefore has a thickness of between 200 and 2000 μm, preferably between 250 and 1800 μm, more preferably between 300 and 1500 μm and even more preferably between 400 and 1200 μm.

In order to ensure a sufficient activity of the catalyst according to the aspects of the invention, the content of Pd in the catalyst is 0.6 to 2.5% by weight, preferably 0.7 to 2.3% by weight and more preferably 0.8 to 2% by weight, relative to the weight of the catalyst support loaded with noble metal.

Furthermore, it may be preferred if the catalyst according to the aspects of the invention has a Pd content of 1 to 20 g/l, preferably 2 to 15 g/l and more preferably 3 to 10 g/l.

Likewise in order to ensure a sufficient activity and selectivity of the catalyst according to the aspects of the invention, the Au/Pd atomic ratio of the catalyst is advantageously between 0 and 1.2, preferably between 0.1 and 1, more preferably between 0.3 and 0.9 and particularly preferably between 0.4 and 0.8.

Moreover, it may be preferred if the Au content of the catalyst according to the aspects of the invention is 1 to 20 g/l, preferably 1.5 to 15 g/l and more preferably 2 to 10 g/l.

In order to ensure a largely uniform activity of the catalyst according to the aspects of the invention across the thickness of the Pd/Au shell, the noble metal concentration should vary only relatively little across the shell thickness. In other words, the profile of the noble metal concentration of the catalyst across an area of 90% of the shell thickness, with the area being spaced apart from the outer and inner shell limit by in each case 5% of the shell thickness, differs from the average noble metal concentration of this area by at most +/−20%, preferably by at most +/−15% and more preferably by at most +/−10%.

Chloride poisons the catalyst according to the aspects of the invention and leads to a deactivation thereof. According to another preferred embodiment of the catalyst according to the aspects of the invention, therefore, its chloride content is less than 250 ppm, preferably less than 150 ppm.

In addition or as an alternative to the dopant oxides mentioned above, the catalyst according to the aspects of the invention may contain at least one alkali metal compound as a further promoter, preferably a potassium, sodium, caesium or rubidium compound, more preferably a potassium compound. Suitable and particularly preferred potassium compounds include potassium acetate KOAc, potassium carbonate K₂CO₃, potassium formate KFA, potassium hydrogen carbonate KHCO₃ and potassium hydroxide KOH and also all potassium compounds which convert into potassium acetate KOAc under the respective reaction conditions of VAM synthesis. The potassium compound may be applied to the catalyst support either before or after the reduction of the metal components to form the metals Pd and Au. According to another preferred embodiment of the catalyst according to the aspects of the invention, the catalyst contains an alkali metal acetate, preferably potassium acetate. In this case, in order to ensure a sufficient promoter activity, it is particularly preferred if the content of alkali metal acetate in the catalyst is 0.1 to 0.7 mol/l, preferably 0.3 to 0.5 mol/l.

According to another preferred embodiment of the catalyst according to the aspects of the invention, the alkali metal/Pd atomic ratio is between 1 and 12, preferably between 2 and 10 and particularly preferably between 4 and 9. Preferably, the smaller the surface area of the catalyst support, the lower the alkali metal/Pd atomic ratio.

The present invention also relates to a first method for the production of a shell catalyst, in particular the shell catalyst according to the aspects of the invention, comprising the steps:
a) providing a porous catalyst support based on a natural sheet-silicate, in particular based on an acid-treated calcined bentonite, said catalyst support being designed as a shaped body, the catalyst support having a surface area of less than 130 m²/g;
b) applying a solution of a Pd precursor compound to the catalyst support;
c) applying a solution of an Au precursor compound to the catalyst support;
d) converting the Pd component of the Pd precursor compound into the metallic form;
e) converting the Au component of the Au precursor compound into the metallic form.

In principle, the Pd and Au precursor compound used may be any Pd or Au compound which makes it possible to achieve a high degree of dispersion of the metals. Here, the term “degree of dispersion” is understood to mean the ratio of the number of surface metal atoms of all metal/alloy particles of a supported metal catalyst relative to the total number of all metal atoms of the metal/alloy particles. In general, it is preferred if the degree of dispersion corresponds to a relatively high numerical value, since in this case the highest possible number of metal atoms are freely accessible for a catalytic reaction. In other words, with a relatively high degree of dispersion of a supported metal catalyst, a certain catalytic activity thereof can be achieved with a relatively low quantity of metal used. According to another preferred embodiment of the catalyst according to the aspects of the invention, the degree of dispersion of the palladium is 1 to 30%.

It may be preferred if the Pd and Au precursor compounds are selected from the halides, in particular chlorides, oxides, nitrates, nitrites, formates, propionates, oxalates, acetates, hydroxides, hydrogencarbonates, amine complexes or organic complexes, for example triphenylphosphine complexes or acetylacetonate complexes, of these metals.

Examples of preferred Pd precursor compounds are water-soluble Pd salts. According to a particularly preferred embodiment of the method according to the aspects of the invention, the Pd precursor compound is selected from the group consisting of Pd(NH₃)₄(OH)₂, Pd(NH₃)₄(OAc)₂, H₂PdCl₄, Pd(NH₃)₄(HCO₃)₂, Pd(NH₃)₄(HPO₄), Pd(NH₃)₄Cl₂, Pd(NH₃)₄ oxalate, Pd oxalate, Pd(NO₃)₂/Pd(NH₃)₄(NO₃)₂, K₂Pd(OAc)₂(OH)₂, Na₂Pd(OAc)₂(OH)₂, Pd(NH₃)₂(NO₂)₂, K₂Pd(NO₂)₄, Na₂Pd(NO₂)₄, Pd(OAc)₂, K₂PdCl₄, (NH₄)₂PdCl₄, PdCl₂ and Na₂PdCl₄, it also being possible to use mixtures of two or more of the aforementioned salts. Instead of NH₃ as ligand, ethyleneamine or ethanolamine may also be used as ligand. Besides Pd(OAc)₂, use may also be made of other carboxylates of palladium, preferably the salts of monocarboxylic acids having 3 to 5 carbon atoms, for example the propionate or butyrate salt.

According to another preferred embodiment of the method according to the aspects of the invention, preference may also be given to Pd nitrite precursor compounds. Preferred Pd nitrite precursor compounds are for example those obtained by dissolving Pd(OAc)₂ in an NaNO₂ solution.

Examples of preferred Au precursor compounds are water-soluble Au salts. According to one particularly preferred embodiment of the method according to the aspects of the invention, the Au precursor compound is selected from the group consisting of KAuO₂, HAuCl₄, KAu(NO₂)₄, NaAu(NO₂)₄, AuCl₃, NaAuCl₄, KAuCl₄, KAu(OAc)₃(OH), HAu(NO₃)₄, NaAuO₂, NMe₄AuO₂, RbAuO₂, CsAuO₂, NaAu(OAc)₃(OH), RbAu(OAc)₃OH, CsAu(OAc)₃OH, NMe₄Au(OAc)₃OH and Au(OAc)₃. It is recommended that the Au(OAc)₃ or the KAuO₂ be freshly prepared in each case by precipitating the oxide/hydroxide out of an auric acid solution, washing and isolating the precipitate and taking up the latter in acetic acid or KOH, respectively.

Suitable solvents for the precursor compounds are all pure solvents or solvent mixtures in which the selected precursor compounds are soluble and which, after application to the catalyst support, can easily be removed again therefrom by drying. Examples of preferred solvents for the metal acetates as precursor compounds are especially unsubstituted carboxylic acids, in particular acetic acid, or acetone, and for the metal chlorides are especially water or dilute hydrochloric acid.

If the precursor compounds are not sufficiently soluble in acetic acid, water or dilute hydrochloric acid or mixtures thereof, other solvents may also be used as an alternative or in addition to the aforementioned solvents. Other solvents which may preferably be mentioned here are those solvents which are inert and can be mixed with acetic acid or water. As preferred solvents which are suitable as an addition to acetic acid, mention may be made of ketones, for example acetone or acetylacetone, and also ethers, for example tetrahydrofuran or dioxane, acetonitrile, dimethylformamide and solvents based on hydrocarbons such as benzene for example.

As preferred solvents or additives which are suitable as an addition to water, mention may be made of ketones, for example acetone, or alcohols, for example ethanol or isopropanol or methoxyethanol, alkaline solutions, such as aqueous KOH or NaOH, or organic acids, such as acetic acid, formic acid, citric acid, tartaric acid, malic acid, glyoxylic acid, glycolic acid, oxalic acid, pyruvic acid, oxamic acid, lactic acid or amino acids such as glycine.

If chloride compounds are used as precursor compounds, it must be ensured that the chloride ions are reduced to a tolerable residual quantity before using the catalyst produced by the method according to the aspects of the invention, since chloride is a catalyst poison. To this end, after the Pd and Au component of the Pd and Au precursor compound has been fixed to the catalyst support, usually the catalyst support is abundantly washed with water. This generally takes place either immediately after the fixing by hydroxide precipitation of the Pd and Au component using an alkaline solution, or after the reduction of the noble metal components to the respective metal/alloy.

However, according to one preferred embodiment of the method according to the aspects of the invention, chloride-free Pd and Au precursor compounds are used along with chloride-free solvents in order to keep the chloride content of the catalyst as low as possible and to avoid any time-consuming washing to remove chloride. In this case, the precursor compounds used are preferably the corresponding acetate, hydroxide, nitrite or hydrogencarbonate compounds, since these contaminate the catalyst support with chloride only to a very limited extent.

The deposition of the Pd and Au precursor compounds on the catalyst support in the region of an outer shell of the catalyst support can be achieved by methods known per se. For instance, the precursor solution can be applied by impregnation, by dipping the support into the precursor solutions or impregnating it in accordance with the incipient wetness method. A base, for example sodium hydroxide solution or potassium hydroxide solution, is then applied to the catalyst support, as a result of which the noble metal components are precipitated out in the form of hydroxides on the support. It is also possible for example to impregnate the support firstly with alkaline solution and then to apply the precursor compounds to the support thus pre-treated.

According to another preferred embodiment of the method according to the aspects of the invention it is therefore provided that the Pd and Au precursor compound is applied to the catalyst support by impregnating the catalyst support with the solution of the Pd precursor compound and with the solution of the Au precursor compound or with a solution which contains both the Pd precursor compound and the Au precursor compound.

According to the prior art, the active metals Pd and Au are applied starting from chloride compounds in the region of a shell of the support to the latter by means of impregnation. However, this technique has reached its limits with regard to minimum shell thicknesses and maximum Au loading. The smallest shell thicknesses of the corresponding known VAM catalysts are at best approximately 100 μm, and it not foreseeable that thinner shells may be obtained by means of impregnation. Furthermore, higher Au loadings within the desired shell by means of impregnation can be achieved only to a very limited extent, since the Au precursor compounds tend to diffuse from the shell into inner zones of the catalyst support shaped body, which leads to wide Au shells which in some regions hardly contain any Pd.

The active metals or the precursor compounds thereof may also be applied to the support, for example by means of so-called physical methods. To this end, the support may according to the aspects of the invention preferably be sprayed with the solution of the precursor compounds, with the catalyst support being moved in a coating drum into which hot air is blown, so that the solvent quickly evaporates.

However, according to one particularly preferred embodiment of the method according to the aspects of the invention, it is provided that the solution of the Pd precursor compound and the solution of the Au precursor compound are applied to the catalyst support by spraying the solutions onto a fluidised bed of the catalyst support, preferably by means of an aerosol of the solutions. In the fluidised bed, the shaped bodies preferably circulate on an elliptical or toroidal course. To give an idea of how the shaped bodies move in such fluidised beds, it may be mentioned that, in the case of an “elliptical circulation”, the catalyst support shaped bodies in the fluidised bed move in the vertical plane on an elliptical course with an alternating size of the main and auxiliary axis.

In the case of “toroidal circulation”, the catalyst support shaped bodies in the fluidised bed move in the vertical plane on an elliptical course with an alternating size of the main and auxiliary axes and in the horizontal plane on a circular course with an alternating size of the radius. On average, the shaped bodies in the case of “elliptical circulation” move in the vertical plane on an elliptical course, and, in the case of “toroidal circulation”, on a toroidal course, which means that a shaped body travels helically over the surface of a torus with an elliptical vertical cross section. As a result, the shell thickness can be smoothly adjusted and optimised, for example up to a thickness of 2 mm. However, very thin shells with a thickness of less than 100 μm are also possible.

The abovementioned embodiment of the method according to the aspects of the invention can be carried out using a fluidised bed system. Particular preference is given to a fluidised bed system in which there is a controlled air slip layer. On the one hand, the catalyst support shaped bodies are thoroughly mixed by the controlled air slip layer, and at the same time rotate about their own axis, as a result of which they are evenly dried by the process air. On the other hand, on account of the resulting orbital movement of the shaped bodies which is brought about by the controlled air slip layer, the catalyst support shaped bodies pass through the spraying process (application of the precursor compounds) at an almost constant rate. This results in a largely uniform shell thickness across a treated batch of shaped bodies. This also means that the noble metal concentration varies only very slightly across a relatively large area of the shell thickness, i.e. that the noble metal concentration across a large area of the shell thickness describes approximately a distorted square-wave function with a high metal concentration on the outside and a somewhat lower metal concentration on the inside, as a result of which a largely uniform activity of the resulting catalyst across the thickness of the Pd/Au shell is ensured.

Suitable coating drums and fluidised bed systems for carrying out the method according to the aspects of the invention in accordance with preferred embodiments are known in the prior art and are sold for example by the companies Heinrich Brucks GmbH (Alfeld, Germany), ERWEK GmbH (Heusenstamm, Germany), Stechel (Germany), DRIAM Anlagenbau GmbH (Eriskirch, Germany), Glatt GmbH (Binzen, Germany), G.S. Divisione Verniciatura (Osteria, Italy), HOFER-Pharma Maschinen GmbH (Weil am Rhein, Germany), L. B. Bohle Maschinen+Verfahren GmbH (Enningerloh, Germany), Lödige Maschinenbau GmbH (Paderborn, Germany), Manesty (Merseyside, United Kingdom), Vector Corporation (Marion, Iowa, USA), Aeromatic-Fielder AG (Bubendorf, Switzerland), GEA Process Engineering (Hampshire, United Kingdom), Fluid Air Inc. (Aurora, Ill., USA), Heinen Systems GmbH (Varel, Germany), Hüttlin GmbH (Steinen, Germany), Umang Pharmatech Pvt. Ltd. (Marharashtra, India) and Innojet Technologies (Lörrach, Germany). Particular preference is given to fluidised bed devices from the company Innojet bearing the name Innojet® Aircoater and Innojet® Ventilus.

According to another preferred embodiment of the method according to the aspects of the invention, the catalyst support is heated during the application of the solutions, for example by means of heated process air. The drying rate of the applied solutions of the noble metal precursor compounds can be determined via the degree of heating of the catalyst support. At relatively low temperatures for example, the drying rate is relatively low, so that, if a suitable quantity is applied, relatively large shell thicknesses can be formed due to the high diffusion of the precursor compounds brought about by the presence of solvents. At relatively high temperatures for example, the drying rate is relatively high, so that any solution of the precursor compounds coming into contact with the shaped bodies dries almost immediately, and therefore solution applied to the catalyst support cannot penetrate deeply into the latter. At relatively high temperatures, therefore, relatively small shell thicknesses with a high noble metal loading can be obtained.

In the methods described in the prior art for the production of VAM shell catalysts based on Pd and Au, use is usually made of commercially available solutions of the precursor compounds such as Na₂PdCl₄, NaAuCl₄ or HAuCl₄ solutions. In more recent literature, as already discussed above, use is also made of chloride-free Pd or Au precursor compounds such as Pd(NH₃)₄(OH)₂, Pd(NH₃)₂(NO₂)₂ and KAuO₂ for example. These precursor compounds react basically in solution, whereas the conventional chloride, nitrate and acetate precursor compounds all react acidically in solution.

In order to apply the precursor compounds to the catalyst support, use is usually preferably made of aqueous Na₂PdCl₄ and NaAuCl₃ solutions. These metal salt solutions are usually applied to the support at room temperature and then the metal components are fixed with NaOH as insoluble Pd or Au hydroxides. The loaded support is then usually washed with water until it is chloride-free. The Au fixing in particular has disadvantages such as long action times of the base to induce the precipitation of the stable Au tetrachloro complex, incomplete precipitation and the associated lack of Au retention.

According to another preferred embodiment of the method according to the aspects of the invention, the method comprises the steps:
a) providing a first solution of a Pd and/or Au precursor compound;
b) providing a second solution of a Pd and/or Au precursor compound, the first solution causing a precipitation of the noble metal component(s) of the precursor compound(s) of the second solution, and vice versa;
c) applying the first and the second solution to the catalyst support.

This embodiment of the method according to the aspects of the invention uses two different precursor solutions, of which one contains a Pd precursor compound and the other contains an Au precursor compound. Preferably, usually one of the solutions has a basic pH and the other has an acidic pH. The solutions are usually applied to the catalyst support by firstly impregnating the support with the first solution and then, in a subsequent step, impregnating it with the second solution as described above by means of soaking. When the second solution is applied, the two solutions are then combined on the support, as a result of which the pH of the solutions changes and the Pd and Au component of the respective precursor compound on the support precipitates out, without having to apply to the support an auxiliary base, such as NaOH or KOH, as is customary in the prior art.

Said embodiment of the method according to the aspects of the invention is therefore based on impregnating the catalyst support with the first solution of a Pd and/or Au precursor compound and the second solution of a Pd and/or Au precursor compound, with the two solutions being incompatible with one another, that is to say that the first solution causes precipitation of the noble metal component(s) of the precursor compound(s) of the second solution and vice versa, so that, in the contact zone of the two solutions, both the pre-impregnated Pd/Au component(s) and the post-impregnated Pd/Au component(s) precipitate out almost simultaneously and thus lead to an intimate Pd/Au mixing. Drying may optionally be carried out between the two impregnation steps.

Suitable aqueous solutions of Pd precursor compounds for impregnation with incompatible solutions are shown by way of example in Table 1.

TABLE 1

| Precursor compound | Nature of the solution |
|---|---|
| PdCl₂ | acidic |
| Pd(NH₃)₂(NO₂)₂ | basic |
| Na₂PdCl₄ | neutral |
| Pd(NH₃)₄(OH)₂ | basic |
| Pd(NO₃)₂ | acidic |
| K₂Pd(OAc)₂(OH)₂ | basic by dissolving |
| | palladium acetate in KOH |

Should NH₃ have an excessively reducing action with regard to premature Au reduction, it is also possible to use, instead of the palladium amine complexes, the corresponding diamine complexes with ethylenediamine as ligand or else the corresponding ethanolamine complexes.

Suitable aqueous solutions of Au precursor compounds for impregnation with incompatible solutions are shown by way of example in Table 2.

TABLE 2

| Precursor compound | Nature of the solution |
|---|---|
| AuCl₃ | acidic |
| KAuO₂ | basic by dissolving Au(OH)₃ |
| | in KOH |
| NaAuCl₄ | neutral |
| HAuCl₄ | acidic |
| KAu(OAc)₃(OH) | basic by dissolving Au(OAc)₃ |
| | in KOH |
| HAu(NO₃)₄ | acidic (stable in semi- |
| | concentrated HNO₃) |

Suitable combinations of incompatible solutions for base-free precipitation of the noble metal components are for example a PdCl₂ solution and a KAuO₂ solution; a Pd(NO₃)₂ solution and a KAuO₂ solution; a Pd(NH₃)₄(OH)₂ solution and an AuCl₃ solution or HAuCl₄ solution.

According to another preferred embodiment of the method according to the aspects of the invention, Pd can also be precipitated with incompatible Pd solutions and similarly Au can be precipitated with incompatible Au solutions, for example by bringing a PdCl₂ solution into contact with a Pd(NH₃)₄(OH)₂ solution or by bringing an HAuCl₄ solution into contact with a KAuO₂ solution. In this way, high Pd and/or Au contents can be deposited in the shell without having to use highly concentrated solutions.

According to another embodiment of the method according to the aspects of the invention, use may also be made of mixed solutions which are compatible with one another and which, for the noble metal precipitation, can be brought into contact with a solution which is incompatible with the mixed solution. An example of a mixed solution is a solution containing PdCl₂ and AuCl₃, the noble metal components of which can be precipitated with a KAuO₂ solution, or a solution containing Pd(NH₃)₄(OH)₂ and KAuO₂, the noble metal components of which can be precipitated with a solution containing PdCl₂ and HAuCl₄. Another example of a mixed solution is the pair HAuCl₄ and KAuO₂.

Impregnation with the incompatible solutions is preferably carried out by means of soaking or by means of spray impregnation, with the incompatible solutions being sprayed on simultaneously through one (dual substance nozzle) or more dual nozzle(s) or simultaneously by means of two nozzles or groups of nozzles or sequentially by means of one or more nozzle(s).

Due to the rapid immobilisation (fixing) of the metal components of the precursor compounds in the shell and the associated shortened Pd and Au diffusion, impregnation with the incompatible solutions may lead to thinner shells than the conventional use of compatible solutions. Using incompatible solutions, it is possible to achieve high noble metal contents in thin shells, improved metal retention, faster and more complete precipitation of the noble metals, a decrease in the disruptive Na residual content of the support, simultaneous fixing of Pd and Au in just one fixing step, and also the elimination of the NaOH costs and NaOH handling and prevention of mechanical weakening of the support through contact with excess NaOH.

By means of impregnation with incompatible solutions, it is possible by just a single fixing step, which includes only the application of two incompatible solutions, for larger noble metal contents to be deposited on the catalyst support than is possible by means of the conventional base (NaOH) fixing.

In particular, using the principle of incompatible solutions, it is easily possible to achieve high Au contents with an Au/Pd atomic ratio of 0.5 and more, which is highly desirable with regard to increasing the VAM selectivity.

According to another preferred embodiment of the method according to the aspects of the invention, it is provided that the catalyst support, once the Pd and/or Au precursor compound(s) has (have) been applied to the catalyst support, is subjected to a fixing step in order to fix the noble metal component(s) of the precursor compound(s) on the catalyst support. The fixing step here may include treatment of the support with an alkaline solution or an acid, depending on whether the precursor compound is acidic or basic, or calcination of the support in order to convert the noble metal component(s) into a hydroxide compound or an oxide. The fixing step may also be omitted and the noble metal components may be reduced directly, for example by treatment with a reducing gas phase, e.g. ethylene, etc. at increased temperatures of 20° C. to 200° C. By means of an intermediate calcination step, the Pd and/or Au precursor compounds can be converted into the oxides and thus fixed.

It is also possible to produce a sheet-silicate-based support material as a powder and to thoroughly impregnate the latter with the precursor compounds of the active metals. The pre-treated powder can then be applied in the form of a “washcoat” to a suitable support structure, for example a sphere made from steatite or a KA-160 support, preferably by means of a coating drum, and then can be further processed by calcination and reduction to form the catalyst.

Accordingly, the invention relates to a second method for the production of a shell catalyst, in particular a shell catalyst according to the aspects of the invention, comprising the steps:
a) providing a pulverulent porous support material based on a natural sheet-silicate, in particular based on an acid-treated calcined bentonite, the support material being loaded with a Pd precursor compound and an Au precursor compound or with Pd and Au particles and having a surface area of less than 130 m²/g;
b) applying the loaded support material to a support structure in the form of a shell;
c) calcining the loaded support structure from step b);
d) optionally, converting the Pd and the Au component of the Pd and Au precursor compound into the metallic form.

As an alternative, said method may also be carried out by firstly applying the pulverulent support material (not loaded with noble metal) to a support structure, and only then applying the noble metals.

Directly after loading with the precursor compounds or after fixing of the noble metal components, the support may be calcined in order to convert the noble metal components into the corresponding oxides. The calcination preferably takes place at temperatures less than 700° C., particularly preferably between 300-450° C., under a supply of air. The calcination time depends on the calcination temperature and is preferably selected within the range of 0.5-6 hours. At a calcination temperature of approximately 400° C., the calcination time is preferably 1-2 hours. At a calcination temperature of 300° C., the calcination time is preferably up to 6 hours. The precipitation fixing may also be omitted and the impregnated salts may be calcined directly in order to convert the metal component into an oxide. One preferred embodiment consists of the (intermediate) calcination of the Pd-loaded support (with or without previous precipitation fixing) at approximately 400° C. in order to form PdO, followed by an Au application and reduction, as a result of which Au sintering can be avoided.

The noble metal components are further reduced before using the catalyst, it being possible for the reduction to be carried out in situ, i.e. in the process reactor, or ex situ, i.e. in a special reduction reactor. Reduction in situ is preferably carried out with ethylene (5% by volume) in nitrogen at a temperature of approximately 150° C. over a time of 5 hours for example. Reduction ex situ may be carried out for example with 5% by volume of hydrogen in nitrogen, for example by means of a forming gas, at temperatures in the range of preferably 150-500° C. over a time of 5 hours.

Gaseous or vaporisable reducing agents such as, for example, CO, NH₃, formaldehyde, methanol and hydrocarbons may also be used, it also being possible for the gaseous reducing agents to be diluted with inert gas, such as carbon dioxide, nitrogen or argon for example. Preferably, a reducing agent diluted with inert gas is used. Preference is given to mixtures of hydrogen with nitrogen or argon, preferably with a hydrogen content of between 1% by volume and 15% by volume.

The reduction of the noble metals may also be carried out in the liquid phase, preferably using the reducing agents hydrazine, K formate, Na formate, ammonium formate, formic acid, K hypophosphite, hypophosphoric acid, H₂O₂ or Na hypophosphite.

The quantity of reducing agent is preferably selected such that, during the treatment time, at least the equivalent required for the complete reduction of the noble metal components is passed over the catalyst. Preferably, however, an excess of reducing agent is passed over the catalyst in order to ensure a rapid and complete reduction.

Reduction is preferably carried out under no pressure, i.e. at an absolute pressure of approximately 1 bar. In order to prepare industrial quantities of catalyst according to the aspects of the invention, use is preferably made of a rotary kiln or a fluidised bed reactor in order to ensure a uniform reduction of the catalyst.

The invention also relates to the use of the catalyst according to the aspects of the invention as an oxidation catalyst, as a hydrogenation/dehydrogenation catalyst, as a catalyst in hydrogenating desulphurisation, as a hydrodenitrification catalyst, as a hydrodeoxygenation catalyst or as a catalyst in the synthesis of alkenyl alkanoates, in particular in the synthesis of vinyl acetate monomer, in particular in the gas phase oxidation of ethylene and acetic acid to form vinyl acetate monomer.

Preferably, the catalyst according to the aspects of the invention is used for the production of VAM. This generally takes place by passing acetic acid, ethylene and oxygen or oxygen-containing gases at temperatures of 100-200° C., preferably 120-200° C., and at pressures of 1-25 bar, preferably 1-20 bar, over the catalyst according to the aspects of the invention, wherein unreacted reagents can be recycled. Advantageously, the oxygen concentration is kept below 10% by volume. In some circumstances, however, dilution with inert gases such as nitrogen or carbon dioxide is also advantageous.

Carbon dioxide is particularly suitable for dilution purposes, since it is formed in small quantities in the course of VAM synthesis. The resulting vinyl acetate is isolated by means of suitable methods, which are described for example in U.S. Pat. No. 5,066,365 A.

The following examples of embodiments, in conjunction with the comparative example, serve to explain the invention:

### EXAMPLE 1

225 g of spherical catalyst support shaped bodies, formed from an acid-treated calcined bentonite as natural sheet-silicate, from the company SÜD-Chemie AG (Munich, Germany) bearing the trade name “KA-0” and having the characteristics shown in Table 3:

TABLE 3

| Geometric shape | sphere | |
|---|---|---|
| Diameter | 5 | mm |
| Moisture content | <2.0% | by weight |
| Compressive strength | >40 | N |
| Bulk density | 528 | g l⁻¹ |
| Water absorbency | 69.5% | |
| Specific surface area (BET) | 106 | m² g⁻¹ |
| SiO₂ content | 95.8% | by weight |
| Al₂O₃ content | 0.95% | by weight |
| Fe₂O₃ content | 0.11% | by weight |
| TiO₂ content | (total) <1.5% by weight | |
| MgO content | | |
| CaO content | | |
| K₂O content | | |
| Na₂O content | | |
| Loss on ignition 1000° C. | <0.2% | by weight |
| Acidity | 50 | μeq/g |
| BJH pore volume N₂ | 0.43 | cm³ g⁻¹ |

were filled into a fluidised bed device from the company Innojet Technologies (Lörrach, Germany) bearing the trade name Innojet® Aircoater and, by means of compressed air (6 bar) heated to 80° C., were brought into a fluidised bed state in which the shaped bodies circulated on a toroidal course, i.e. moved on a vertically oriented ellipsoidal path and on a horizontal circular path oriented perpendicular thereto.

Once the shaped bodies had been brought to a temperature of approximately 75° C., 300 ml of an aqueous mixed noble metal solution containing 7.5 g of commercially available Na₂PdCl₄ (sodium tetrachloropalladate) and 4.6 g of commercially available NaAuCl₄ (sodium tetrachloroaurate) were sprayed onto the fluidised bed of shaped bodies over 40 min.

Once the catalyst support had been impregnated with the mixed noble metal solution, a 0.05 molar NaOH solution at a temperature of 80° C. was sprayed onto the fluidised bed of shaped bodies over 30 min. During this, most of the NaOH is deposited within the shell and fixes the Pd and Au metal components, without the support being exposed to excessively high NaOH concentrations.

After the NaOH has acted, the supports were abundantly washed with water in the fluidised bed device in order to remove from the supports most of the alkali metal and chloride that had been introduced into the support via the noble metal compounds and NaOH.

After washing, the shaped bodies were dried in the fluidised bed device by moving them in hot process air (100° C.)

Once the shaped bodies had been dried, they were reduced with a gas mixture of ethylene (5% by volume) in nitrogen at a temperature of approximately 150° C. in the fluidised bed device to form a Pd/Au shell catalyst.

The resulting shell catalyst contained approximately 1.2% by weight of Pd and had an Au/Pd atomic ratio of approximately 0.5, a shell thickness of approximately 160 μm and a hardness of 38 N.

Across an area of 90% of the shell thickness, with the area being spaced apart from the outer and inner shell limit by in each case 5% of the shell thickness, the noble metal concentration of the Pd/Au shell catalyst thus produced, differed from the average noble metal concentration of this area by at most +/−10%. The noble metal distribution was determined using a scanning electron microscope LEO 430VP, equipped with an energy dispersive spectrometer from the company Bruker AXS. In order to measure the noble metal concentration across the shell thickness, a catalyst sphere was cut through, glued to an aluminium sample holder and then vaporised with carbon. The detector used was a nitrogen-free silicon drift detector (XFlash® 410) with an energy resolution of 125 eV for the manganese Kₐₗₚₕₐ line.

### EXAMPLE 2

65.02 g of catalyst support shaped bodies “KA-0” as defined in Example 1 are impregnated with 43.8 ml of an aqueous solution containing 1.568 g of Na₂PdCl₄ and 0.367 g of HAuCl₄ according to the pore filling method (incipient wetness method), in which a support is impregnated with a volume of solution corresponding to its pore volume. After the impregnation, 89.17 g of a 0.35 molar NaOH solution are added to the catalyst support shaped bodies and the latter are left to stand overnight at RT for 22 hours. After decanting off the fixing solution, the catalyst precursor thus produced is reduced with 73.68 g of a 10% NaH₂PO₂ solution (Fluka) for 2 hours. After draining off the reducing solution, the catalysts are washed with distilled water for 8 hours at RT with continuous replacement of the water (throughflow=140 rpm) in order to remove Cl residues. The final value for the conductivity of the washing solution is 1.2 μS.

Thereafter, the catalyst is dried in the fluidised bed at 90° C. for 50 min. The dried spheres are loaded with a mixture of 27.29 g of 2 molar KOAc solution and 18.55 g of H₂O and are left to stand for one hour at room temperature. Finally, drying is carried out for 40 min at 90° C. in the fluidised bed.

The theoretical metal loading is 0.8% by weight of Pd and 0.3% by weight of Au; the values determined experimentally by elemental analysis using ICP (Inductively Coupled Plasma) were 0.77% by weight of Pd and 0.27% by weight of Au.

The shell thickness was 312 μm.

### Comparative Example 1

A catalyst was prepared in the same way as in Example 2, with a support from the company SUD-Chemie AG bearing the trade name “KA-160” and having the characteristics shown in Table 4 being used as the catalyst support shaped bodies:

TABLE 4

| Geometric shape | sphere | |
|---|---|---|
| Diameter | 5 | mm |
| Moisture content | <2.0% | by weight |
| Compressive strength | >60 | N |
| Bulk density | 554 | g l⁻¹ |
| Water absorbency | 62% | |
| Specific surface area (BET) | 158 | m² g⁻¹ |
| SiO₂ content | 93.2% | by weight |
| Al₂O₃ content | 2.2% | by weight |
| Fe₂O₃ content | 0.35% | by weight |
| TiO₂ content | (total) <1.5% by weight | |
| MgO content | | |
| CaO content | | |
| K₂O content | | |
| Na₂O content | | |
| Loss on ignition 1000° C. | <0.3% | by weight |
| Acidity | 53 | μeq/g |
| BJH pore volume N₂ | 0.38 | cm³ g⁻¹ |

By contrast to Example 2, impregnation was carried out with 39.1 ml of an aqueous solution containing 1.568 g of Na₂PdCl₄ and 0.367 g of HAuCl₄.

The theoretical metal loading is 0.8% by weight of Pd and 0.3% by weight of Au; the values determined experimentally by elemental analysis using ICP were 0.78% by weight of Pd and 0.27% by weight of Au.

The shell thickness was 280 μm.

### EXAMPLE 3

Reactor Test

6 ml of a fill of catalyst spheres of Example 2 and of Comparative Example 1 were in each case acted upon by a feed gas stream of 550 Nml/min composed of 15% HOAc, 6% O₂, 39% C₂H₄ in N₂ in a fixed bed tube reactor at a temperature of 150° C. at 10 bar, and the reactor discharge was analysed by means of gas chromatography.

The selectivity (from ethylene to VAM) is calculated according to the formula S(C₂H₄)=mole VAM/(mole VAM+mole CO₂/2). The space/time yield is obtained as g VAM/l catalyst/h. The oxygen conversion is calculated by (mole O₂ in-mole O₂ out)/mole O₂ in.

The catalyst of Example 2 according to the aspects of the invention exhibits a selectivity S(C₂H₄) of 92.3% and a space/time yield (determined by gas chromatography) of 615 g VAM/l catalyst/h for an oxygen conversion of 36.5%.

The catalyst of Comparative Example 1 exhibited a selectivity S (C₂H₄) of 91.0% and a space/time yield (determined by gas chromatography) of 576 g VAM/l catalyst/h for an oxygen conversion of 36.1%.

The catalyst of Example 2 according to the aspects of the invention exhibits both a higher selectivity and also activity in VAM synthesis compared to a catalyst of the prior art according to Comparative Example 1.

1. A shell catalyst for the production of vinyl acetate monomer, comprising a porous catalyst support based on a natural sheet silicate, in particular based on an acid-treated calcined bentonite, said catalyst support being loaded with Pd and Au and being designed as a shaped body, wherein the catalyst support has a surface area of less than 130 m²/g.

2. The catalyst according to claim 1, wherein the catalyst support has a surface area of less than 125 m²/g, preferably less than 120 m²/g, more preferably less than 100 m²/g, even more preferably less than 80 m²/g and particularly preferably less than 65 m²/g.

3. The catalyst according to claim 1, wherein the catalyst support has a surface area of between 130 and 40 m²/g, preferably of between 128 and 50 m²/g, more preferably of between 126 and 50 m²/g, even more preferably of between 125 and 50 m²/g, still more preferably of between 120 and 50 m²/g and most preferably of between 100 and 60 m²/g.

4. The catalyst according to claim 1, wherein the catalyst support has an acidity of between 1 and 150 μeq/g, preferably of between 5 and 130 μeq/g, more preferably of between 10 and 100 μeq/g and particularly preferably of between 10 and 60 μeq/g.

5. The catalyst according to claim 1, wherein the catalyst support has an average pore diameter of 8 to 50 nm, preferably 10 to 35 nm and more preferably 11 to 30 nm.

6. The catalyst according to claim 1, wherein the catalyst has a hardness of greater than/equal to 20 N, preferably greater than/equal to 30 N, more preferably greater than/equal to 40 N and most preferably greater than/equal to 50 N.

7. The catalyst according to claim 1, wherein the proportion of natural sheet silicate, in particular of acid-treated calcined bentonite, of the catalyst support is greater than/equal to 50% by weight, preferably greater than/equal to 60% by weight, more preferably greater than/equal to 70% by weight, even more preferably greater than/equal to 80% by weight, still more preferably greater than/equal to 90% by weight and most preferably greater than/equal to 95% by weight, relative to the weight of the catalyst support.

8. The catalyst according to claim 1, wherein the catalyst support has an integral pore volume according to BJH of between 0.25 and 0.7 ml/g, preferably between 0.3 and 0.6 ml/g and more preferably from 0.35 to 0.5 ml/g.

9. The catalyst according to claim 1, wherein at least 80% of the integral pore volume of the catalyst support is formed of mesopores and macropores, preferably at least 85% and more preferably at least 90%.

10. The catalyst according to claim 1, wherein the catalyst support has a bulk density of more than 0.3 g/ml, preferably more than 0.35 g/ml and particularly preferably a bulk density of between 0.35 and 0.6 g/ml.

11. The catalyst according to claim 1, wherein the sheet silicate contained in the support has an SiO₂ content of at least 65% by weight, preferably at least 80% by weight and more preferably from 95 to 99.5% by weight.

12. The catalyst according to claim 1, wherein the sheet silicate contained in the support contains less than 10% by weight of Al₂O₃, preferably 0.1 to 3% by weight and more preferably 0.3 to 1.0% by weight.

13. The catalyst according to claim 1, wherein the catalyst support is shaped as a sphere, cylinder, perforated cylinder, triple lobe, ring, star or as strand, preferably as a ribbed strand or star-shaped strand, preferably as a sphere.

14. The catalyst according to claim 1, wherein the catalyst support is shaped as a sphere having a diameter of more than 2 mm, preferably having a diameter of more than 3 mm and more preferably having a diameter of more than 4 mm.

15. The catalyst according to claim 1, wherein the catalyst support is doped with at least one oxide of a metal selected from the group consisting of Zr, Hf, Ti, Nb, Ta, W, Mg, Re, Y and Fe, preferably with ZrO₂, HfO₂ or Fe₂O₃.

16. The catalyst according to claim 15, wherein the content of dopant oxide in the catalyst support is between 0.01 and 20% by weight, preferably 1.0 to 10% by weight and more preferably 3 to 8% by weight.

17. The catalyst according to claim 1, wherein the shell of the catalyst has a thickness of less than 300 μm, preferably less than 200 μ, more preferably less than 150 μm, even more preferably less than 100 μm and still more preferably less than 80 μm.

18. The catalyst according to claim 1, wherein the shell of the catalyst has a thickness of between 200 and 2000 μm, preferably between 250 and 1800 μm, more preferably between 300 and 1500 μm and even more preferably between 400 and 1200 μm.

19. The catalyst according to claim 1, wherein the content of Pd in the catalyst is 0.6 to 2.5% by weight, preferably 0.7 to 2.3% by weight and more preferably 0.8 to 2% by weight, relative to the weight of the catalyst support loaded with noble metal.

20. The catalyst according to claim 1, wherein the Au/Pd atomic ratio of the catalyst is between 0 and 1.2, preferably between 0.1 and 1, more preferably between 0.3 and 0.9 and particularly preferably between 0.4 and 0.8.

21. The catalyst according to claim 1, wherein the noble metal concentration of the catalyst across an area of 90% of the shell thickness, with the area being spaced apart from the outer and inner shell limit by in each case 5% of the shell thickness, differs from the average noble metal concentration of this area by at most +/−20%, preferably by at most +/−15% and more preferably by at most +/−10%.

22. The catalyst according to claim 1, wherein the catalyst has a chloride content of less than 250 ppm, preferably less than 150 ppm.

23. The catalyst according to claim 1, wherein the catalyst comprises an alkali metal acetate, preferably potassium acetate.

24. The catalyst according to claim 23, wherein the content of alkali metal acetate in the catalyst is 0.1 to 0.7 mol/l, preferably 0.3 to 0.5 mol/l.

25. The catalyst according to claim 23, wherein the alkali metal/Pd atomic ratio is between 1 and 12, preferably between 2 and 10 and more preferably between 4 and 9.

26. A method for the production of a shell catalyst, in particular a shell catalyst according to claim 1, comprising the steps:
a) providing a porous catalyst support on the basis of a natural sheet silicate, in particular the basis of an acid-treated calcined bentonite, said catalyst support being designed as a shaped body, wherein the catalyst support has a surface area of less than 130 m²/g;
b) applying a solution of a Pd precursor compound to the catalyst support;
c) applying a solution of an Au precursor compound to the catalyst support;
d) converting of the Pd component of the Pd precursor compound into the metallic form;
e) converting the Au component of the Au precursor compound into the metallic form.

27. The method according to claim 26, wherein the Pd and Au precursor compounds are selected from the halides, in particular chlorides, oxides, nitrates, nitrites, formates, propionates, oxalates, acetates, hydroxides, hydrogencarbonates, amine complexes or organic complexes, for example triphenylphosphine complexes or acetylacetonate complexes, of these metals.

28. The method according to claim 26, wherein the Pd precursor compound is selected from the group consisting of Pd(NH₃)₄(OH)₂, Pd(NH₃)₄(OAc)₂, H₂PdCl₄, Pd(NH₃)₄(HCO₃)₂, Pd(NH₃)₄(HPO₄), Pd(NH₃)₄Cl₂, Pd(NH₃)₄ oxalate, Pd(NO₃)₂, Pd(NH₃)₄(NO₃)₂, K₂Pd(OAc)₂(OH)₂, Pd(NH₃)₂(NO₂)₂, K₂Pd(NO₂)₄, Na₂Pd(NO₂)₄, Pd(OAc)₂, PdCl₂ and Na₂PdCl₄.

29. The method of claim 26, wherein the Au precursor compound is selected from the group consisting of KAuO₂, HAuCl₄, KAu(NO₂)₄, AuCl₃, NaAuCl₄, KAu(OAc)₃(OH), HAu(NO₃)₄, NaAuO₂, NMe₄AuO₂, RbAuO₂, CsAuO₂, NaAu(OAc)₃(OH), RbAu(OAc)₃OH, CsAu(OAc)₃OH, NMe₄Au(OAc)₃OH and Au(OAc)₃.

30. The method of claim 26, wherein the Pd and Au precursor compound is applied to the catalyst support by impregnating the catalyst support with the solution of the Pd precursor compound and with the solution of the Au precursor compound or with a solution which contains both the Pd precursor compound and the Au precursor compound.

31. The method of claim 26, wherein the solution of the Pd precursor compound and the solution of the Au precursor compound is applied to the catalyst support by spraying the solutions onto a fluidized bed or fluid bed of the catalyst support, preferably by means of an aerosol of the solutions.

32. The method of claim 26, wherein the catalyst support is heated during the application of the solutions.

33. The method of claim 26, wherein
a) a first solution of a Pd and/or Au precursor compound is provided;
b) a second solution of a Pd and/or Au precursor compound is provided, wherein the first solution causes a precipitation of the noble metal component(s) of the precursor compound(s) of the second solution, and vice versa;
c) the first and the second solutions are applied to the catalyst support.

34. The method of claim 33, wherein the precursor compounds of one solution are acidic and those of the other solution are basic.

35. The method of claim 26, wherein the catalyst support, once the Pd and/or Au precursor compound(s) has (have) been applied to the catalyst support, is subjected to a fixing step.

36. A method for the production of a shell catalyst, in particular a shell catalyst according to claim 1, comprising the steps:
a) providing a pulverulent porous support material on the basis of a natural sheet silicate, in particular on the basis of an acid-treated calcined bentonite, wherein the support material is loaded with a Pd precursor compound and an Au precursor compound or with Pd and Au particles and having a surface area of less than 130 m²/g;
b) applying the loaded support material to a support structure in the form of a shell;
c) calcining the loaded support structure of step b);
d) optionally, converting the Pd and the Au component of the Pd and Au precursor compound into the metallic form.

37. Use of a catalyst according to claim 1 as an oxidation catalyst, as a hydrogenation/dehydrogenation catalyst, as a catalyst in hydrogenating desulphurisation, as a hydrodeoxygenation catalyst, as a hydrodenitrification catalyst or as a catalyst in the synthesis of alkenyl alkanoates, in particular in the synthesis of vinyl acetate monomer, in particular in the gas phase oxidation of ethylene and acetic acid to vinyl acetate monomer.
